(19) United States
(12) Patent Application Publication
(10) Pub. No.: US 2004/0091390 A1
(43) Pub. Date: May 13, 2004

(54) Method for removal of mold and other biological contaminants from a surface

(76) Inventor: Jeffrey B. Bentley, Corpus Christi, TX (US)

Correspondence Address: GUNN, LEE & HANOR, 700 N. ST. MARY'S STREET, SUITE 1500, SAN ANTONIO, TX, 78205, US

(21) Appl. No.: 10/292,361

(22) Filed: Nov. 12, 2002

(51) Int. Cl. A61L 9/00

(52) U.S. Cl. 422/28; 405/5; 405/30; 405/32; 405/34

(57) ABSTRACT

A method for killing and removing mold and other biological contaminants from structural surfaces. The contaminated surface is enclosed and isolated in a containment area from other noncontaminated surfaces. A biocidal agent is applied to the contaminated surface to kill the contaminant. After the biocidal agent dryes, the contaminant is encapsulated. The contaminant and encapsulant are then removed from the surface by propelling frozen carbon dioxide pellets against the surface at a high velocity. The removed contaminants are collected and disposed of with a high volume air extractor equipped with filters. The containment area may be treated with ultraviolet radiation to kill any remaining contaminants which may be airborne or adhere to the containment area surfaces. A biocidal sealer may be applied to the formerly contaminated surface to prevent regrowth of the contaminant. After the contaminant removal process is completed, air quality testing is performed to insure that the air quality in the containment area meets with applicable environmental standards.

### BACKGROUND OF THE INVENTION

1. Field of The Invention

The present invention relates to a method for the removal of mold, fungi, bacteria, and other biological contaminants from surfaces and disposal of the same. More specifically, this invention combines the use of biocidal agents and encapsulating agents with dry ice blasting, germicidal UV lighting, and various vacuuming techniques to remove mold and other biological contaminants from building's walls, floors, ceilings, and other surfaces.

2. Background Information

There are several methods used in the art for the removal of mold and other biological contaminants from surfaces of buildings or other structures. The most common methods include removal of the structural element containing the mold on its surface or removal of the surface mold by sanding, wire brushing, scraping, planing, and/or water blasting. While these methods may be effective for removing mold spores on the surface, they fail to reach mold growing in cracks and crevices and other hard to reach areas. They also fail to adequately remove the thin layer of decomposed substrate that is food source for the mold. This layer contains the brown hyphae and mycelial fragments (roots) that will rebloom as mold when moisture or humidity returns. Viable mold spores can also become airborne during the processes, causing health risks and additional mold growth on nearby surfaces. In addition, these processes are cumbersome and time consuming. In the case of water blasting, an excessive amount of contaminated water is created which can be difficult to dispose of and acts as a catalyst for the regrowth of the mold.

Other methods for the removal of mold or other biological contaminants include using heavy applications of harsh biocidal chemicals such as chlorine dioxide. An example of the use of chlorine dioxide is disclosed in the patent to Sumner A. Barenberg and Peter N. Gray, U.S. Pat. No. 5,980,826. Still other practitioners in the art use ozone shock treatments by introducing ozone into the environment surrounding the contaminated surface. The ozone acts to oxidize and kill the biological contaminant. The use of chemicals is effective for killing the mold spores on the contaminated surface. However, it does not remove the mold or effectively kill the thin layer of hyphae and mycelial fragments which will, once again, rebloom. In addition, the use of heavy doses of harsh chemicals releases toxic fumes into environment surrounding the contaminated surface causing potential health risks. In the case of the ozone shock treatments, the presence of ozone can cause plastic surfaces to deteriorate.

It is also common in the art to combine the heavy use of harsh chemicals or ozone shock treatments with the mechanical removal of the mold by first chemically treating the contaminated surface to kill the active mold spores and then mechanically removing the mold by sanding, wire brushing, scraping, or planing. Once again, the combination of these methods does not effectively remove the layer of decomposed substrate containing the hyphae and mycelial fragments. In the case of using a wire brush to clean structures such as wood studs, the bristles follow the path of least resistance and follow the wood grain cleaning primarily down into the valleys of the softer spring growth rings leaving the sides contaminated.

A patent to Hedmon, U.S. Pat. No. 6,327,812 describes a method for destroying organisms and toxins in an enclosure such as a building to include fungi and toxic molds. This patent discloses directing an environmentally acceptable gas, such as air, into an enclosure and heating it to a temperature sufficiently high to kill the viable mold. The enclosure is equipped with ingress and egress ducts that create a flow of air through the enclosure in an attempt to remove dead organisms. This method is ineffective for removing the mold from the contaminated surface and also ineffective in eliminating the thin layer of substrate containing the hyphae and mycelial fragments.

Carbon dioxide ice blasting for cleaning surfaces is known in the art and currently there are several dry ice blasting machines commercially available. Carbon dioxide ice blasting involves propelling carbon dioxide particles against a surface and it has several advantages. For example, it can remove and clean unwanted material from a surface without damaging the substrate. Moreover, the carbon dioxide particles sublime upon contact and dissipate into the atmosphere leaving only the removed material for disposal. Heretofore, carbon dioxide blasting has not been used for removing mold and other biological contaminants from surfaces.

It is desirable to have a new method which more effectively kills, removes, disposes of, and prevents the regrowth of mold and other biological contaminants on building surfaces.

### SUMMARY OF THE INVENTION

It is an object of this invention to provide a method for killing mold and other biological contaminants without the heavy use of biocidal chemicals.

It is another object of this invention to provide a method for effectively killing airborne and surface mold spores without the use of ozone shock treatments.

It is a further object of this invention to provide an easier and less time consuming method for removing mold and other biological contaminants from building surfaces.

It is another object of this invention to provide a method for removing mold and other biological contaminants from cracks, crevices, and other hard to reach areas on building surfaces.

It is another object of this invention to provide for a method for the removal of mold and other biological contaminants which prevents regrowth of the same.

It is a further object of this invention to provide a method for the removal of mold and other biological contaminants which allows for the easy and efficient disposal of removed contaminant particles and at the same time having little or no need for disposal of the cleaning agent.

It is another object of this invention to provide for a method of removing mold which also removes the thin layer of substrate which contains the brown hyphae and mycelial fragments.

It is a further object of this invention to provide a method of mold and biological contaminant removal from building surfaces which reduces the number of airborne mold spores and other biological contaminant particles during the mechanical removal process.

In order to achieve these objectives, this invention provides a method which comprises the following steps.

First, the contaminated surface is contained and sealed off from other noncontaminated surfaces and thereby creating a contained environment. The contaminated surface is then exposed by removing walls, flooring, tiles, fixtures, etc. The containment area is equipped with air extractors to create negative air throughout the containment area. The contaminated surfaces are then treated with a light concentration of a chemical biocide and allowed to dry.

Next, the contaminated surface is treated with a penetrating encapsulant which, in the case of mold, penetrates and bonds the mold spores, hyphae, and mycelia into a larger encapsulated mass. Once it has been encapsulated, the mold or biological contaminated surface is then blasted cleaned with carbon dioxide particles. The temperature and force of the dry ice particles causes the encapsulated mass to harden, crack, and break off from the surface leaving a clean contaminant free substrate. The contaminant particles which are broken off, become airborne and caught in the airflow and removed from the containment area via high volume air extractors. The containment area and contaminated surface is then vacuumed with a HEPA (High Efficiency Particle Arrestor) vacuum. The HEPA vacuum is equipped with a HEPA filter that removes 99.97% of particulates with a size of 0.3 microns or larger.

In order to ensure that the newly cleaned surfaces do not “rebloom”, the containment area is exposed to a shortwave, ultraviolate radiation from a germicidal UV light to kill any remaining mold spores, hyphae, or biological contaminants that may be present. The newly cleaned surface is then coated and sealed with a co-polymer acrylic/ polyacrylate type fungicidal sealer applied to the surface.

Once the sealant has dried, high volume fans equipped with filters (air scrubbers) are introduced into the containment area and all contaminant surfaces are agitated with air blowers while the air scrubbers are continuously running and circulating the air. Any mold spores or other biological contaminants that are not sealed will be removed by this step. The air blower can be any commercially available leaf blower or similar equipment.

Once the air scrubbing process is complete, the air quality in the containment area is tested to see if it meets with EPA or local environmental mold standards. If the air quality test passes, the containment area barriers are removed and the previously contained area is now ready for remodeling if necessary. If the air quality does not pass, the following steps are repeated: the contaminant area is retreated with a light concentrations of biocidal agent; the containment area is vacuumed with a HEPA vacuum; the containment area is disinfected with UV radiation; the newly cleaned surface is resealed with another coating of fungicidal sealer; and the containment area is air scrubbed. After these steps are repeated, the air quality in containment area is, once again tested. If the air quality test does not pass, these steps are repeated until it does.

### BRIEF DESCRIPTION OF THE DRAWINGS

FIG. 1 is a block diagram depicting the steps of the preferred embodiment.

FIG. 2 is a schematic depicting the first and second steps of the preferred embodiment.

FIG. 3 is a schematic depicting the fifth step of the preferred embodiment.

FIG. 4 is a schematic depicting the sixth step of the preferred embodiment.

FIG. 5 is a schematic depicting the ninth step of the preferred embodiment.

FIG. 6 is a schematic depicting the tenth step of the preferred embodiment.

### DETAILED DESCRIPTION OF THE PREFERRED EMBODIMENT

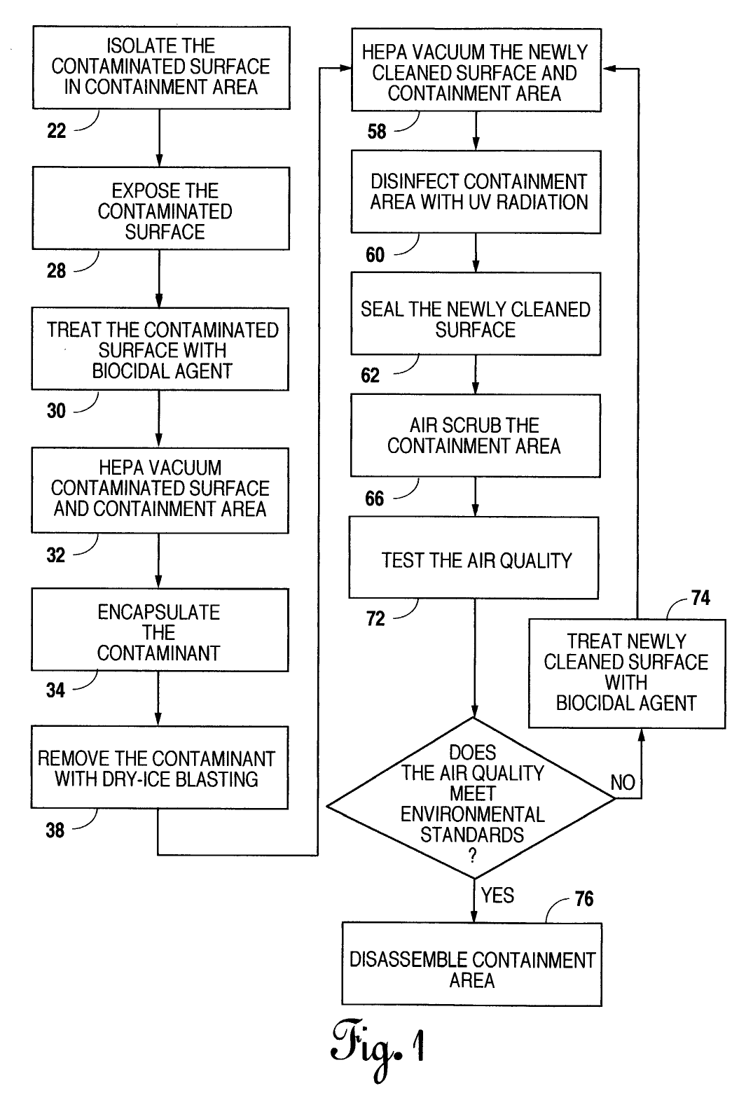

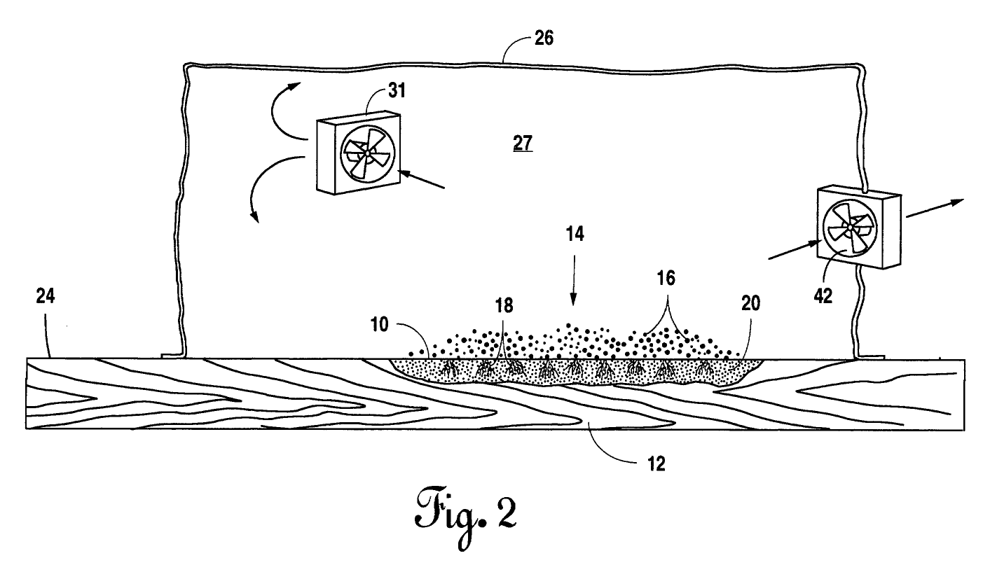

Reference is now made to FIGS. 1 and 2 for a detailed description of the steps appropriate for implementing the method of the present invention. In FIG. 2, a structural element 12 has a contaminated surface 10 covered with mold 14. The mold 14 is comprised of surface mold spores 16 and hyphae 18. The hyphae extend into a layer of decomposed soft wood 20 which acts as a food source for the mold 14.

The first step 22 (see FIGS. 2.1 and 2) of the present invention is to isolate the contaminated surface 10 from noncontaminated surfaces 24 of the structural element 12 by erecting a negative air pressure barrier 26 around the contaminated surface 10 and sealing it off from the noncontaminated surfaces 24 and thereby creating a containment area 27. In the preferred embodiment, the negative air pressure barrier 26 is made of a flexible plastic material and the containment area 27 is spacious enough to allow workers to work within along with the equipment as outlined in the following steps.

Continuing the first step, as shown in FIG. 2, negative air is established in the containment area 27 using an air extractor 29 positioned at the negative air pressure barrier 26 that moves air outside the containment area 27. The air extractor 29 is a fan which draws air through a filter. in the preferred embodiment, the air extractor 29 is manufacture by Force Air, model number FA2000EC and is equipped with three filters. The first filter removes particles up to 10 microns and larger. The second filter removes particles up to 1 micron and larger and typically has an efficiency rating of 30% at 1 micron. The third filter is a HEPA filter and has an efficiency rating of 99.97% at 0.3 microns. The first two filters act to prevent the premature loading of the third, more expensive HEPA filter. It is anticipated that other similar, commercially available air extracting units can be substituted.

Continuing the first step, an air scrubber 31 is introduced into the containment area 27 to circulate and filter the air within the containment area 27. The air scrubber 31 is also a fan which draws air through a filter. In the preferred embodiment, the air scrubber 31 is identical to the air extractor 29. It is anticipated that other similar, commercially air scrubbing units could be substituted. The air extractor 29 and the air scrubber 31 operate and move air continuously throughout the remaining steps of the method of the present invention.

In the second step 28 (see FIG. 1) of the present invention, the contaminated surface 10 is exposed for preparation for the removal process. This second step 28 involves removing walls, flooring, tiles, fixtures, etc. to expose the structural element 12 with the contaminated surface 10. Any damaged structural materials are bagged and disposed of.

In the third step 30 (see FIG. 1) of the present invention, the contaminated surface 10 is sprayed with a light concentration of a biocidal agent to kill the surface mold spores 16. The biocidal agent is applied with an airless spray gun or fogger until the contaminated surface 10 is damp and then allowed to dry. In the preferred embodiment, the biocidal agent is a 2% active chlorine dioxide gas in a stabilized solution. However, it is anticipated that the biocidal agent may be one or a combination of biocides such as Clorox bleach, Oxine, Alcide, Wavicide, Sporocidin, Sporax, Quate solutions, Amphyl, or other similar EPA approved agents.

In the fourth step 32 (see FIG. 1), all surfaces in the containment area 27 are vacuumed with a HEPA vacuum to remove mold spores and other mold particles which have adhered to the surfaces in the containment area 27. The HEPA vacuum is equipped with a HEPA filter with a 99.97% retention efficiency at 0.3 microns. The HEPA filter is sufficient to trap the average mold spore which is 4 microns. In the preferred embodiment, the HEPA vacuum is manufactured by Nilfisk, Advance Model No. 137. When the vacuuming is completed the HEPA filters are removed, bagged, and disposed of.

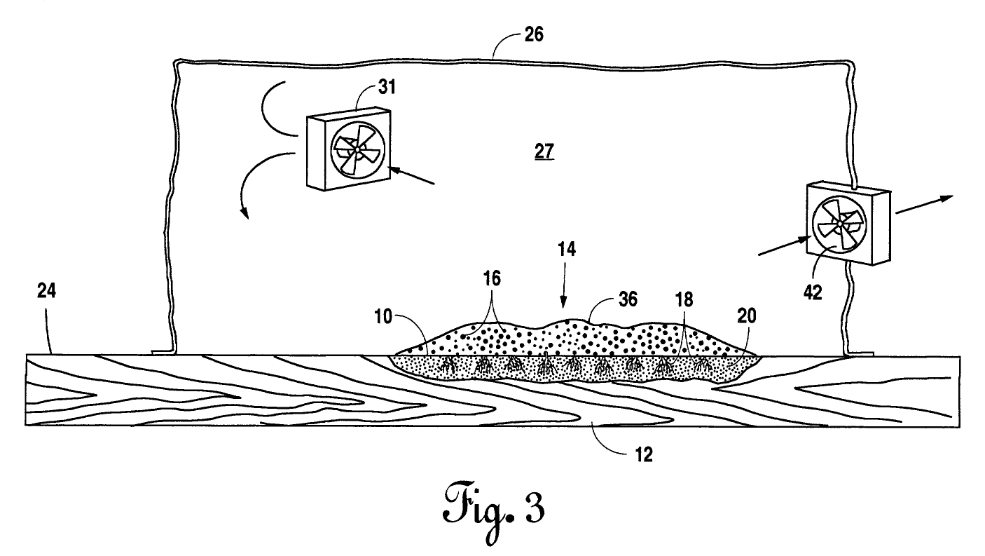

In the fifth step 34 (see FIG. 1), the mold 14 is treated with a penetrating encapsulant. As shown in FIG. 3, the encapsulant coats and penetrates the surface mold spores 16 and the layer of decomposed soft wood 20 and bonds the surface mold spores 16, hyphae 18, and the layer of decomposed soft wood 20 into an encapsulated mass 36.

In the fifth step 34, the penetrating encapsulant is applied with a high output electric airless spray gun, pneumatic/hydraulic spray equipment or other similar spraying equipment. In the preferred embodiment, the pressure of the spraying equipment is set as low as possible while still achieving atomization of the liquid encapsulant and still achieving coating and penetration of the mold 14 and the layer of decomposed soft wood 20. The penetrant/encapsulant can be a Sodium Silicate Solution (silici acid, sodium salt and water) which has a ph of around 11.3 and may be diluted with 5-10% water. This agent ph will kill mold which grows in a ph of around 5-6 for slightly acidic environment. In other embodiments, Foster products 40-20 series, Fiberlock 7000-8000 series or other similar products may be used as the penetrant/encapsulant.

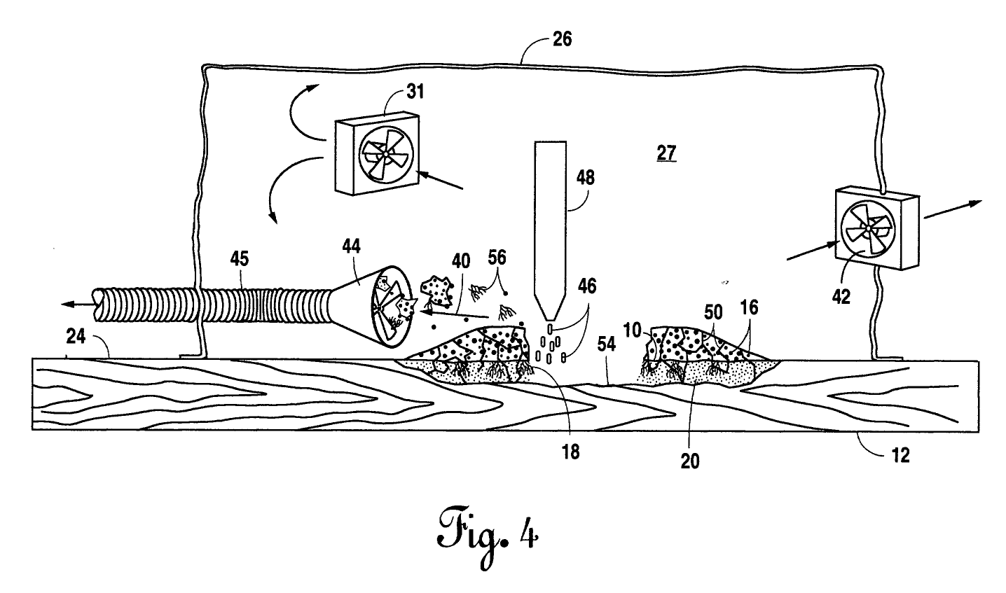

Once the encapsulated mass 36 has been created, the mold, hyphae, and layer of decomposed soft wood forming the encapsulated mass is removed in the sixth step 38 (see FIG. 1). A shown in FIG. 4, the encapsulated mass 36 is bombarded with frozen carbon dioxide particles 46 being propelled at a high velocity from dry ice blasting equipment 48. In the preferred embodiment, the dry ice blasting equipment 48 is the Alpheus SDI-mini blast or similar equipment with pressure and ice volume control.

Still referring to FIG. 4, the frozen carbon dioxide particles 46 have a temperature of −78.5° C., which causes cracks 50 in the encapsulated mass 36 as it cools and hardens under the cold temperature. The force of the frozen carbon dioxide particles 46 and the blast air from the dry ice blasting machine 48 causes the encapsulated mass 36 to break off in clumps 52 which are carried away from the contaminated surface 10 by the kinetic energy of the frozen carbon dioxide particles 46 and the blast air. A high volume air extractor 44, capable of moving 5,000 to 7,000 cfm of air, is placed near the blast point and operates continuously during the sixth step 38 to carry the airborne clumps 52 and mold and hyphae dust particles 56 out of the contaminant area. The high volume air extractor 44 is equipped with a hose 45 of a length and diameter sufficient to allow the high volume air extractor to be moved as the blast point is moved and to carry the airborne clumps 52 and the mold and hyphae dust particles 56 out of the containment area. The high volume air extractor 44 is equipped with a HEPA filter which traps the clumps 52 and loose mold and hyphae dust particles 56. Once the dry ice blasting is completed, the HEPA filters are removed, bagged, and disposed of. In the preferred embodiment, the filter is a HEPA filter which removes 99.97% retention efficiency at 0.3 microns. In the preferred embodiment the high volume air extractor is manufactured by ______ , model number ______ . However, it is anticipated that other similar commercial available air extractors could be used.

Still referring to FIG. 4, the dry ice blasting continues until all of the encapsulated mass 36, clumps 52, loose mold and hyphae dust particles 56, and the layer of decomposed soft wood 20 are removed from the structural element 12 leaving a newly cleaned surface 54. In the preferred embodiment the blast pressure of the dry ice blasting machine 48 should be between 40 and 75 PSI with a dry ice feed rate of between ½ lb. and 1 lb. per minute. Upon impact with the encapsulated mass 36, the frozen dioxide particles 46 sublime, leaving a cloud of carbon dioxide gas which is carried away with the airflow 40 through the high volume air extractor 44. This cloud of super cooled carbon dioxide gas is heavier than air in the contaminant area 27 and helps to contain the clumps 52 and loose mold and hyphae dust particles 56 in the immediate blast area. This allows for more effective removal of the clumps 52 and loose mold and hyphae dust particles 56 from the containment area via the airflow 40 through the high volume air extractor 44.

The air used in the dry ice blasting machine is first compressed using a Kaeser air compressor, model Mobilair 120T or any other similar commercially available air compressors. The compressed air is then passed through a chilling/drying unit which cools the compressed air to room temperature and lowers its moisture content. Cooling the air ensures that the frozen carbon dioxide particles 46 (see FIG. 4) do not prematurely sublime. Lowering the moisture content of the air helps retard mold growth by drawing moisture from the contaminated surface during the blasting process. In the preferred embodiment, the chiller/drying unit is manufactured by Kaeser Compressors, Model KAD260. However, it is anticipated that other similar, commercially available chillers and dryers could be used.

Referring again to FIG. 1, the seventh step 58 of the present invention occurs after the mold 14 and the layer of decomposed soft wood 20 has been completely removed from the structural element 12 by the blasting process. In the seventh step 58, all of the surfaces in the containment area 10 are, once again, vacuumed with a HEPA vacuum which is equipped with a HEPA filter with a 99.97% retention efficiency at 0.3 microns. When the vacuuming is complete, the HEPA filters are removed, bagged, and disposed of.

In the eighth step 60 (see FIG. 1) of the present invention, in order to disinfect the containment area, shortwave ultraviolet radiation is introduced into the containment area 27 at a level of 30,000 microwatt-seconds per square centimeter at 254 nm. At this level the shortwave, UV radiation is lethal to molds, fungi, and bacteria. The ultraviolet radiation is introduced with the use of germicidal UV lamps such as the Sanidyne portable area sanitizer manufactured by Atlantic Ultraviolet Corporation or other similar commercially available UV lamps.

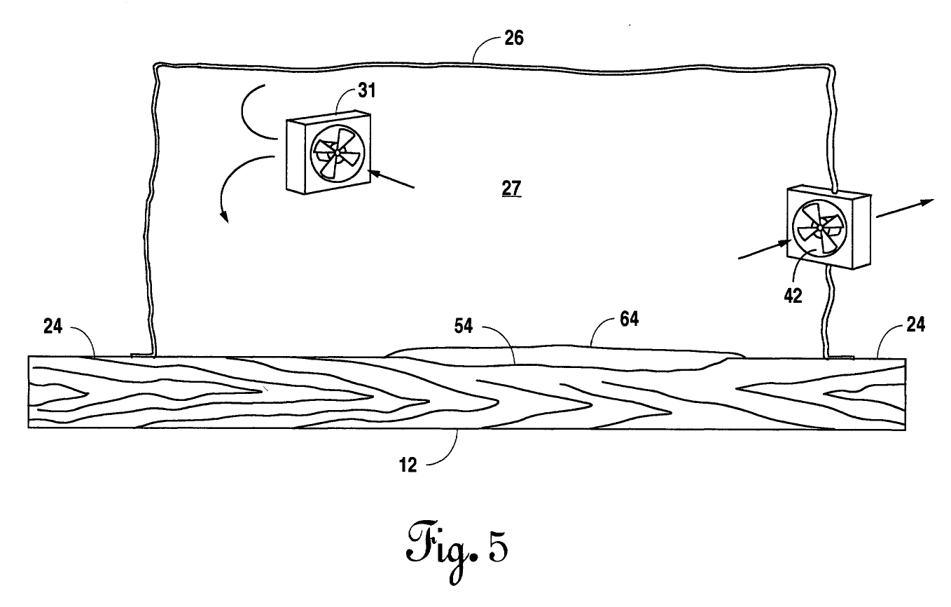

In the ninth step 62 (see FIG. 1) of the method of the present invention, the newly cleaned surface 54 is coated and sealed with a fungicidal protective coating 64 as shown in FIG. 5. Foster Products 40-20 series or Fiberlock 7000-8000 series are suitable for this purpose. However, other equivalent fungicidal sealers can be used. The fungicidal protective coating 64 prevents any remaining surface mold spores 16 from becoming airborne and creates a fungicidal barrier for any future mold growth. In the preferred embodiment, the fungicidal protective coating 64 is applied with an airless spray gun. However, it is anticipated that other methods of application can be used.

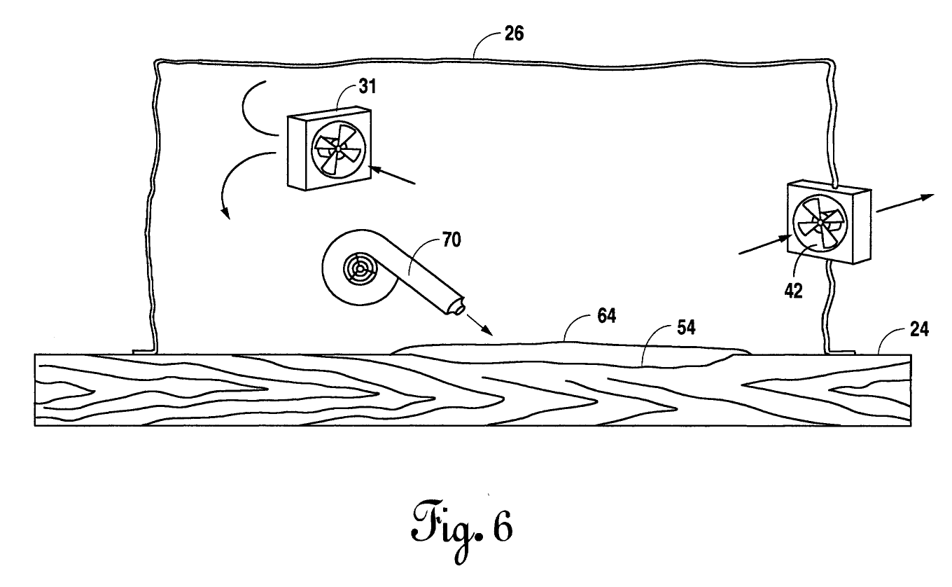

In the tenth step 66 (see FIGS. 1 and 6) of the present invention, the air in the containment area is filtered and cleaned one final time. To accomplish this, the air extractor 29 and air scrubber 31 are allowed to run continuously for a period of three days. However, this length of time of operation can vary with the size of the containment area, and the volume of air the air scrubber can circulate. It is preferable to circulate the entire volume of air in the containment area at least one time ever 10 minutes.

Still referring to FIG. 6, during the air scrubbing process, all surfaces of the containment area 27 are periodically agitated with an air blower unit 70. This causes any mold spores or other biological contaminants which have adhered to the surfaces of the containment area 27 to become airborne and circulate through the air scrubber 68. In the preferred embodiment, the surfaces of the containment area 27 are agitated three times per day during the air scrubbing process. It is anticipated that the air blower can be any commercially available leaf blower of other similar air-blowing device.

In the eleventh step 72 (see FIG. 1) of the present invention, the air in the containment area 27 is tested to see if the air quality meets EPA or local environmental mold standards. In order to perform the test, airborne fungal spore samples are collected by drawing air calibrated with a Dwyer Series VFB Visa-Float Flowmeter at approximately 15 liters per minute through the industry standard air-o-cell cassettes.

As shown in FIG. 1, the method of the present invention moves on to the twelfth step 74 if the test results show that the air quality does not meet EPA or local environmental standards. In the twelfth step 74, the newly cleaned surface 54 is once again sprayed with a light concentration of a biocidal agent as described in the third step 30 of the present invention and then seventh step 58 through the eleventh step 72 are repeated. In repeating the air scrubbing process of the tenth step 66, the air scrubber 68 is run continuously for one day. If, in repeating the eleventh step 72, the test results show that the air quality, once again does not meet EPA or local environmental standards, the twelfth step 74 and the seventh step 58 through the eleventh step 72 are repeated until the air quality test passes.

Once again referring to FIG. 1, the method of the present invention moves on to step thirteen 76 when the air quality tests conducted in the eleventh step meets environmental standards. In step thirteen 76, the barrier 26 forming the containment area 27 and all equipment are removed. All negative air pressure filters or damaged structure materials which have not been previously disposed of are bagged and disposed of.

Although the method of the invention has been described with reference to specific embodiments, this description is not meant to be construed in a limited sense. Various modifications of the disclosed embodiments a well as alternative embodiments of the invention will become apparent to persons skilled in the art upon the reference to the description of the invention. It is, therefore, contemplated that the appended claims will cover such modifications that will fall within the scope of the invention.

I claim:

1. A method for the removal of biological contaminant from a contaminated surface comprising the steps of:
exposing said contaminated surface;
propelling frozen carbon dioxide particles against said contaminated surface at a high velocity, said frozen carbon dioxide particles being propelled in fluid medium;
freezing said biological contaminant with the contact of said frozen carbon dioxide particles;
removing said biological contaminant from said contaminated surface with the force of said frozen carbon dioxide particles and said fluid medium.

2. The method of claim 1 wherein said biological contaminant is mold.

3. The method of claim 1 wherein said fluid medium is compressed gas.

4. The method of claim 3 wherein said compressed gas is compressed air.

5. The method of claim 4 wherein said compressed air is cooled to room temperature before being used as said fluid medium for said propelling of said frozen carbon dioxide particles against said contaminated surface.

6. The methods of claims 4 or 5 wherein said compressed air is dried prior to being used as said medium for said propelling of said frozen carbon dioxide particles against said contaminated surface.

7. The method of claim 1 further comprising a first step of isolating within a containment area said contaminated surface from noncontaminated surfaces before exposing said contaminated surface.

8. The method of claim 7 further comprising the steps of:
collecting said biological contaminants removed from said contaminated surface; and
disposing of said biological contaminants removed from said contaminated surface.

9. The method of claim 8 wherein said collecting of said biological contaminant removed from said contaminated surface is accomplished with an air extraction device which draws air inside said containment area containing said biological contaminants removed from said contaminated surface into said air extraction device.

10. The method of claim 9 wherein said disposal of said biological contaminant removed from said contaminated surface is accomplished by said air extraction device which carries said biological contaminants removed from said contaminated surface outside said containment area.

11. The method of claim 9 wherein said air extraction device is equipped with a filter or a series of filters, said filter(s) trapping said biological contaminant removed from said contaminated surface.

12. The method of claim 11 wherein said disposal of said biological contaminants removed from said contaminated surface is accomplished by periodically bagging and disposing of said filters or series of filters.

13. A method for the removal of biological contaminants from a contaminated surface comprising the steps of:
exposing said contaminated surface;
encapsulating said biological contaminant by applying a penetrating, encapsulating agent to said contaminated surface;
removing said biological contaminant from said contaminated surface by propelling frozen carbon dioxide particles against said encapsulating agent on said contaminated surface thereby breaking up said encapsulating agent containing said biological contaminants.

14. The method of claim 13 wherein said biological contaminant is mold.

15. The method of claim 14 further comprising a first step of isolating within a containment area surrounding said contaminated surface from noncontaminated surfaces before exposing said contaminated surface.

16. The method of claim 15 further comprising the step of creating negative air pressure in said contaminant area relative to the air pressure outside said containment area prior to exposing said contaminated surface.

17. The method of claim 16 wherein said negative air pressure is accomplished by continuously extracting air from said containment area.

18. The method of claim 17 further comprising the steps of:
killing said biological contaminant on said contaminated surface after exposing said contaminated surface by applying a light application of a biocidal agent to said contaminated surface until said contaminated surface is damp; and
allowing said biocidal agent to dry prior to said encapsulating of said biological contaminant.

19. The method of claim 18 further comprising the step of vacuuming all surfaces of said containment area after said biocidal agent has dried but before removing said biological contaminant from said contaminated surface.

20. The method of claim 19 further comprising the steps of:
collecting said biological contaminants removed from said contaminated surface; and
disposing of said biological contaminants removed from said contaminated surface.

21. A method for the removal of biological contaminants from a contaminated surface comprising the steps of:
isolating within a containment area said contaminated surface from noncontaminated surfaces;
exposing said contaminated surface;
removing said biological contaminant from said contaminated surface by propelling frozen carbon dioxide particles against said contaminated surface; and
disinfecting said containment area.

22. The method of claim 21 wherein said biological contaminant is mold.

23. The method of claim 22 wherein said isolating of said contaminated surface is accomplished by erecting plastic barriers surrounding said contaminated surface.

24. The method of claim 23 further comprising the step of sealing said contaminated surface after said disinfecting of said containment area.

25. The method of claim 24 further comprising the step of circulating said air inside said containment area through an air scrubber.

26. The method of claim 25 further comprising the step of testing the quality of said air inside said containment area to see if said air quality meets selected environmental standards.

27. The method of claim 26 further comprising the following steps if said testing of said air quality indicates said air quality fails to meet said selected environmental standards:
vacuuming all said surfaces of said containment area;
repeating said disinfecting step;
repeating said sealing step;
repeating said air circulating step; and
repeating said testing step.

28. The method of claim 27 wherein the steps as recited in claim 27 are repeated if said air quality inside said containment area fails to meet said selected environmental standards.

29. The method of claim 21 wherein said disinfecting step is accomplished with ultraviolet radiation.
